(19) United States
(12) Patent Application Publication
(10) Pub. No.: US 2003/0073971 A1
(43) Pub. Date: Apr. 17, 2003

(54) Universal connector and method for securing flexible tube assemblies

(76) Inventor: Gordon Mark Saker, Haleiwa, HI (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 09/981,585

(22) Filed: Oct. 17, 2001

(51) Int. Cl. A61B 19/00

(52) U.S. Cl. 604/403

(57) ABSTRACT

An universal connector is disclosed. The universal connector can be realized with a housing having an inlet port, an outlet port, and a tapered inner surface; and a restrictor fixedly attached to the housing. In some exemplar embodiments, the universal connector is configured with a slot for receiving a tubing coupler. The universal connector may be realized in methods for securely coupling flexible tubing sections associated in a tubing assembly. A preferred method includes the steps of: (1) selecting an appropriately configured universal connector; (2) inserting a first end of a tubing junction within the housing; and (3) axially rotating a second end of the tubing junction until the tubing assembly junction is substantially aligned with the longitudinal axis of the connector.

### TECHNICAL FIELD

The present invention relates generally to flexible tube assemblies, and more particularly, to an apparatus and method for securing medical tube junctions.

### BACKGROUND

In the field of medicine, plastic tubing is in common use for various purposes. One such purpose lies in transferring fluids to or from the human body, or directly within the body. In all such uses, it is generally necessary to use special connectors or fittings to connect lengths of tubing together, to connect tubes to fittings, etc. It is also generally necessary and desirable that the connections be easy to make, that they be made quickly, and that they be reliable. This is especially true when the tube sections and connectors are used to transfer fluids to a mobile patient such as in the application of a gastric feeding system with a pediatric patient.

Gastric tubes have been used to administer essential nutrient solutions and medications to patients with severe dysphagia and/or other medical conditions. The gastric tube is inserted through a hole, or gastronomy, through the abdominal wall and the stomach wall, to directly introduce the solutions and medications into a patient's stomach. The terms “gastric tubes,” “gastronomy tubes,” and “enteral feeding tubes” are used interchangeably in this specification. For the purposes of this specification, enteral means of, relating to, or being a medicinal preparation treated to pass through the stomach unaltered and disintegrate in the intestines.

Feeding solutions and/or medications are introduced via an assembly constructed of a supply and various flexible-tube sections having one or more fittings or couplers connected to a gastronomy tube. The feeding solutions and/or medications may be gravity fed to an enteral feeding pump which may be adjusted as required to obtain a desired ingestion rate. Generally, the enteral feeding pumps use a rotating cam that engages a portion of the supply tube. Each rotation of the cam transfers a known volume of the feeding solution and/or medication from the supply portion of the tube assembly to the gastronomy tube coupled to the patient. By controlling the rotational speed of the cam, the enteral feeding pump supplies a known volume of solution and/or medication over a specified treatment period.

Silicone tubes are extensively applied in enteral feeding systems because they are easy to manufacture and compatible with feeding solutions and medications administered therein. Silicone tubes are also used in a host of internal applications because of their compatibility with the human body. Silicone tubes, however, must be handled with care. In particular, it has been observed that silicone tubes have a tendency to fail when over stretched, subjected to sharp corners, or deformed such that their tensile yield strength is exceeded.

A number of tube section couplers presently in use include a generally conical hollow male member. The couplers have an input port over which a first section of soft flexible tubing is stretched over to form a first connection with the coupler. A second section of flexible tubing is stretched over the conical and tapered exit port to form a second or exit connection with the coupler. These couplers suffer from the weakness that when used with certain types of flexible tubes such as silicone tubes, the second connection is subject to failure in tension since the tubes are often over stretched when placed about the conical male member.

Furthermore, silicone tubes have a tendency to neck inward when subjected to axial stretching forces. Such axial stretching forces are commonly encountered in enteral feeding assemblies associated with pediatric patients in the toddler stage. This inward necking tends to help the tubing pull loose from the coupler.

In some other assemblies, the male coupler is inserted into a fitting. These tubing assemblies rely on a friction fit between the outer surface of the male coupler and the inner surface of an orifice of the fitting. These assemblies suffer from the weakness that in some cases a significant portion of the surface area of the male coupler is not in contact with the fitting. Consequently, the assembly suffers from a single point of failure at assembly junctions constructed in this manner. Because of these problems, mechanical couplings and similar fittings are finding increasing use in medical applications to connect various lengths of tubing. A significant number of mechanical couplers suitable for connecting two sections of flexible tubing are directed to the problem of contamination protection. These mechanical couplers are typically complex and expensive to manufacture. Often these mechanical couplers include multiple couplers, clamps, clips, and the like, which tend to pinch one or both tubes causing subsequent failure of the assembly due to the eventual failure of the tube.

Moreover, while it is certainly undesirable to permit possible contamination of the feeding solutions and medications transferred via an enteral feeding system, it is just as important to ensure that a mobile patient, such as a pediatric patient is unable to pull the enteral feeding tube assembly apart, resulting in messy spillage and inherent waste of the feeding solution and medication. Of even greater importance to the patient and to the patient's caregivers is the resulting unknown loss of feeding time and improper dosages of medications that result from enteral feeding assembly failures and subsequent attempts to estimate and correct for the volumetric loss. While it is technically possible to recover and measure the volume of the spilled fluids, it is difficult at best and highly impractical to expect caregivers to accurately correct for the amount of fluid lost during an enteral feeding tube assembly failure. For patients with a particularly sensitive digestive system that can only receive a limited volume of feeding solution over a specified time or for patients that absorb a limited amount of nutrients regardless of the volume of the feeding solution provided, an enteral feeding assembly failure can result in an unrecoverable opportunity for life sustaining nourishment.

For at least these reasons, it is desirable to provide a low cost, highly effective alternative solution that overcomes the shortcomings of the prior art.

### SUMMARY OF THE INVENTION

In light of the foregoing, a universal connector is introduced. The universal connector can be realized with a housing having an inlet port, an outlet port, and a tapered inner surface; and a restrictor fixedly attached to the housing, the restrictor configured to engage a second end of the tubing junction. When the universal connector is configured with a housing that closely contacts the external surface of a stretched tubing section that overlaps the external surface of the coupler, the tubing assembly is prevented from disengaging. In some exemplar embodiments, the universal connector is configured with a slot for receiving a tubing coupler.

The universal connector may be realized in methods for securely coupling flexible tubing sections associated in a tubing assembly. A preferred method includes the steps of: (1) selecting an appropriately configured universal connector; (2) inserting a first end of a tubing junction within the housing; and (3) axially rotating a second end of the tubing junction until the tubing assembly junction is substantially aligned with the longitudinal axis of the connector.

Variations of the apparatus, methods, and features herein presented will be or become apparent to one with skill in the art upon examination of the following drawings and detailed description. All variations are included within the scope of the universal connector as described by the accompanying claims.

### BRIEF DESCRIPTION OF THE DRAWINGS

The accompanying drawings incorporated in and forming a part of the specification illustrate several aspects of the universal connector, and together with the description serve to explain the principles thereof. The components in the drawings are not necessarily to scale, emphasis instead placed upon clearly illustrating the principles of the universal connector. Moreover, in the drawings, like reference numerals designate corresponding parts throughout the several views. In the drawings:

FIG. 1 is a schematic diagram illustrating an exemplar-tubing junction susceptible to failure due to disengagement.

FIG. 2 is a schematic diagram illustrating the various assemblies of a second exemplar-tubing junction susceptible to failure due to disengagement.

FIG. 3 is a schematic diagram illustrating an operative position of the tubing junction of FIG. 2. FIG. 4 is a schematic diagram illustrating a top view of a preferred embodiment of the universal connector of the present invention.

FIG. 5 is a schematic diagram illustrating a side view of the universal connector of FIG. 4.

FIG. 6 is a schematic diagram illustrating a perspective view of a universal connector consistent with the present invention.

FIG. 7 is a schematic diagram illustrating the universal connector of FIG. 4 in operative arrangement with the exemplar-tubing junction of FIG. 1.

FIG. 8 is a schematic diagram illustrating a variation of the universal connector of the present invention in operative arrangement with the exemplar-tubing junction of FIG. 3.

FIGS. 9A-9C are a series of schematic diagrams illustrating a variation of the universal connector of the present invention in top, side, and front views, respectively.

FIG. 10 is a flowchart illustrating a method for keeping coupled flexible tubing sections in a tubing assembly from disengaging that may be practiced by the universal connector of the present invention.

### DETAILED DESCRIPTION

Having summarized the invention above, reference is now made in detail to the description of the universal connector as illustrated in the drawings. While the universal connector will be described in connection with these drawings in the context of enteral feeding systems, there is no intent to limit it to the embodiment or embodiments disclosed therein. On the contrary, the intent is to cover all alternatives, modifications, and equivalents included within the spirit and scope defined by the appended claims.

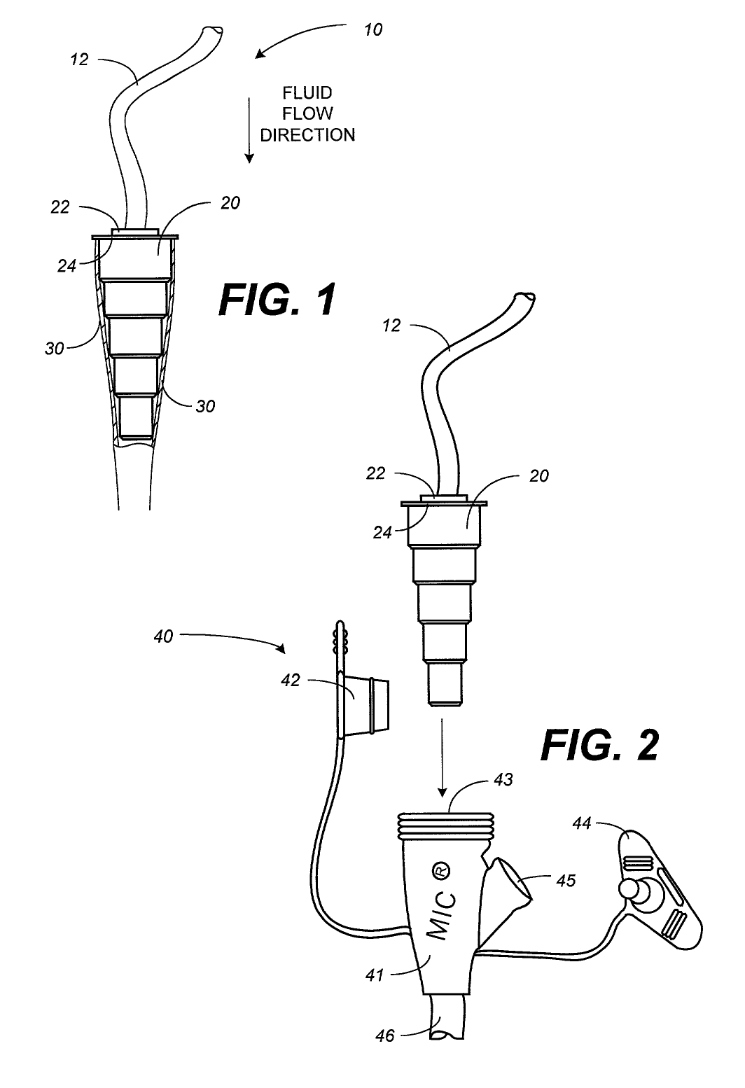

The tubing junction illustrated in FIG. 1 is offered by way of example to reveal a common tubing junction between a first tubing section and a second tubing section that is susceptible to failure due to disengagement of the stretched (i.e., the second tubing section) from a male coupler. Such a tubing junction can be found in the commercially offered Kangaroo Pet™ Enteral Feeding Kit supplied by the Sherwood Medical Supply Company of St. Louis, Mo. As illustrated in FIG. 1, a tubing assembly 10 comprises a first tubing section 12, a tapered male coupler 20, and a second tubing section 30. As shown by the fluid flow direction arrow in the schematic, fluids including various feeding solutions and liquid based medications flow through the hollow first tubing section 12 through the tapered male coupler 20 to the stretched and partially overlapping second tubing section 30. As described above, this and other similarly constructed tubing assemblies are susceptible to disengagement due to tension forces along the fluid flow direction (i.e., along the longitudinal axis of the tapered male coupler 20).

As shown in FIG. 1, the tapered male coupler 20 consists of a hollow multi-step taper with each of the subsequent steps having a progressively smaller diameter from an initial portion closest to a leading edge of the second tubing section 30. As is also shown in FIG. 1, the tapered male coupler 20 is configured with an inlet port 22 and a plate 24. The inlet port 22 is generally configured with a nipple having an external diameter appropriately selected such that it closely engages the interior surface of the first tubing section 12. In some embodiments, the junction formed at the inlet port 22 may be strengthened by lengthening the nipple along the longitudinal axis of the coupler 20 and/or by adding a suitable adhesive to either or both contact surfaces of the first tubing section or the outer surface of the nipple.

Plate 24, as shown in FIG. 1, is configured with a diameter that protects the leading edge of the second tubing section 30. Stated another way, the plate 24 extends beyond the leading edge of the second tubing section 30 such that the leading edge is not susceptible to snagging or separation from the initial or largest diameter portion of the tapered male coupler 20.

The tubing junction illustrated in FIG. 2 is offered by way of example to reveal a second common tubing junction between a first tubing section and a second tubing section that is susceptible to failure due to disengagement of a second (non-stretched) tubing section from a male coupler. Such a tubing junction can be found in the combination of the tubing junction of FIG. 1 and the commercially offered Low Profile Gastronomy Kit marketed as the MIC-KEY® G™ as supplied by the Medical Innovations Corporation a division of Ballard Medical Products of Draper, Utah.

As illustrated in FIG. 2, a tubing assembly 40 comprises a first tubing assembly consisting of a subset of the tubing assembly 10 of FIG. 1 (i.e., the tubing assembly 10 without the “stretched” second tubing section 30). As shown by the downwardly pointing arrow of FIG. 2, the first tubing assembly, may be inserted into Y-shaped fitting 41. The Y-shaped fitting 41 includes a main inlet port 43, a secondary inlet port 45, and an outlet tube 46. As is further illustrated in FIG. 2, the Y-shaped fitting 41 may be adapted to receive multiple feeding and medicinal solutions, or alternatively a single solution via either of the inlet ports 43, 45. A single input solution can be chosen by selectively closing one of the inlet ports 43, 45 with its associated plug 42, 44, respectively.

As with the tubing assembly 10 of FIG. 1, fluids including various feeding solutions and liquid based medications flow through the hollow first tubing section 12 through the tapered male coupler 20 and into the primary inlet port 43 of the Y-shaped fitting 41. As described above, this and other similarly constructed tubing assemblies are also susceptible to disengagement due to forces applied to the outlet tube 46 where the forces have a component in the fluid flow direction.

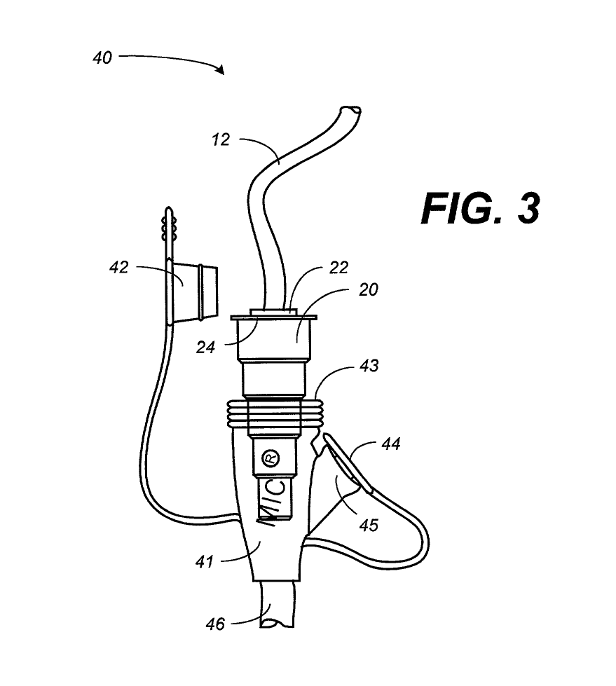

Reference is now directed to FIG. 3, which illustrates the various assemblies of FIG. 2 coupled in an exemplar operative position. As illustrated in FIG. 3, the partial tubing assembly 10 of FIG. 1 is engaged in the primary inlet port 43 of the Y-shaped fitting 41. As further illustrated in FIG. 3, the secondary inlet port 45 is closed by plug 44.

As shown in FIG. 3, the Y-shaped fitting 41, and in fact most of the tubing assembly components used in medical applications, are constructed of translucent materials to provide an indication of proper fluid flow. In some cases, couplers and/or relatively short sections of tubing may be formed of opaque materials. For example, the tapered male coupler 20 in the Kangaroo Pet™ Enteral Feeding Kit is constructed of an opaque plastic. The tubing assembly 40, illustrated in FIG. 3, is susceptible to disengagement due to forces applied on the Y-shaped fitting 41 and/or the outlet tube 46. This is particularly true because a significant portion of multi-step tapered end of the male coupler 20 is not engaged within the primary inlet port 43 of the Y-shaped fitting 41.

Universal Connector

FIG. 4 is a schematic diagram illustrating a top view of a preferred embodiment of the universal connector of the present invention. In this regard, the universal connector 100 includes a restrictive surface 110, a housing 120, an inlet port 112, and an outlet port 130. In this particular embodiment, the restrictive surface 110 forming the inlet port 112 is configured such that the inlet port 112 is shaped to closely contact the outer surface of a first section of tubing 12 (i.e., a supply tube) associated with the tubing assembly 10 of FIG. 1. In fact, in some arrangements, the restrictive surface 110 forming the inlet port 112 is shaped such that the restrictive surface 110 compresses a portion of the outer surface of the first section of tubing 12. Note that the restrictor 110 in the present embodiment is shaped such that it surrounds a substantial portion of the outer surface of the first section of tubing 12 thereby aligning the tubing assembly with the longitudinal axis of the universal connector 100.

As illustrated in FIG. 4, the housing 120 may be configured such that it forms an aperture 122 for receiving the tubing assemblies 10, 40 of FIGS. 1 and 3, respectively. The housing 120 may be configured with a taper such that the inner surface of the housing 120 engages the various surfaces of the tubing assemblies 10, 40 of FIGS. 1 and 3. In addition, the housing 120 may form a slot 124 for passively receiving and retaining the second section of tubing 30, 46 of FIGS. 1 and 3, respectively. As shown in FIG. 4, the slot 124 may be formed such that it is receives and releases the tubing assemblies along the longitudinal axis of the universal connector 100.

Furthermore, the outlet port 130 of the universal connector 100 formed by the housing 120 is configured such that it substantially surrounds the outer diameter of the second section of tubing 30, 46 of FIGS. 1 and 3. In this way, the housing 120 in addition to receiving and engaging the tubing assemblies 10, 40 (FIGS. 1 and 3) also serves to align the tubing assemblies with the longitudinal axis of the universal connector 100. Consequently, tubing assemblies 10, 40 are secured from failing due to disengagement from tension forces in the fluid flow direction applied by the patient and/or external sources. Tubing assemblies 10, 40 are also secured from failing due to disengagement from axial forces that may applied to the various components of the assemblies.

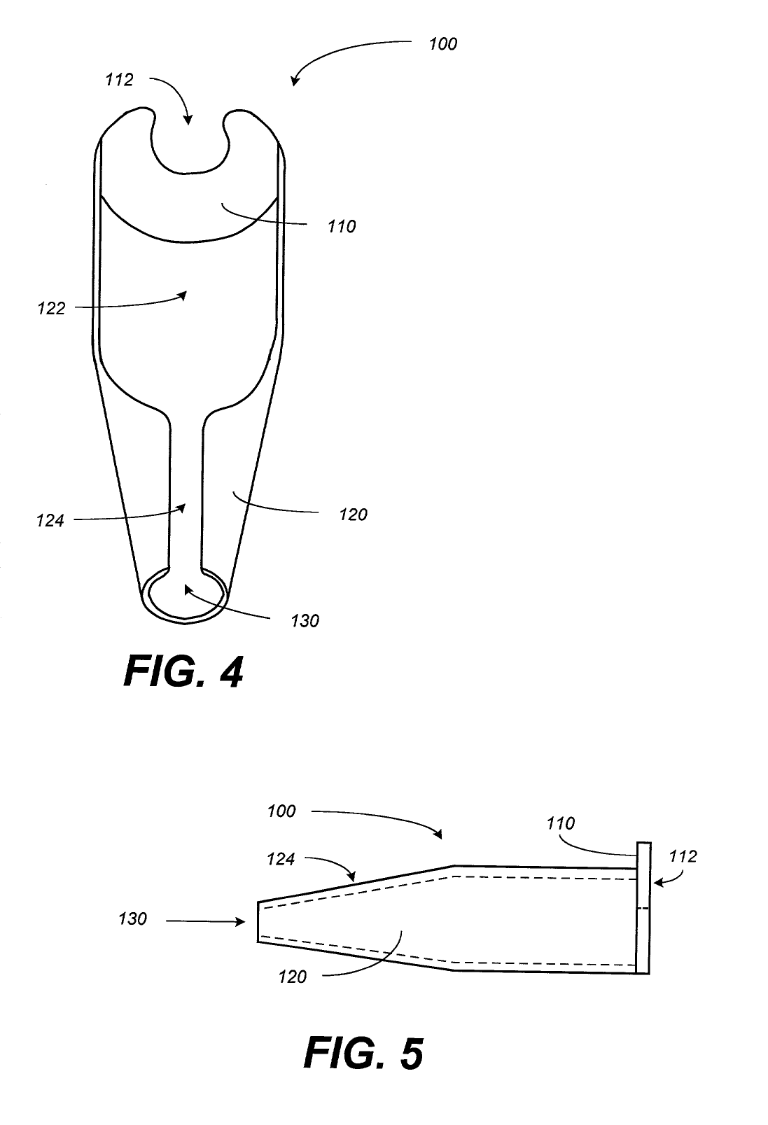

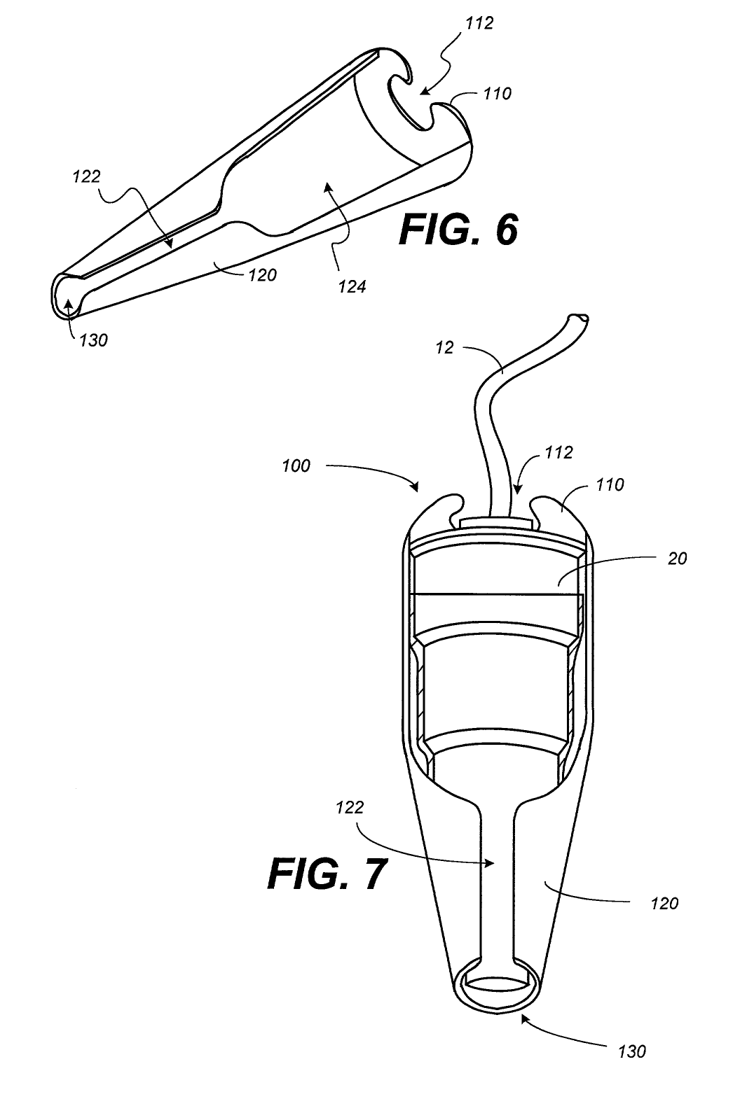

Reference is now directed to FIG. 5, which presents a side view of the universal connector of FIG. 4. In this regard, the illustration details the relationship of the various components of the universal connector 100. Looking at the universal connector 100 from left to right, the universal connector includes the exit port 130 and slot 124 formed by the housing 120. At the extreme right of the figure, the universal connector includes a restrictor 110, which forms the inlet port 112 of the universal connector 100. It is significant to note that the angle of the taper of the housing 120, the diameter of the outlet port 130, the size and shape of the slot 124, the size and shape of the restrictor 110 and the size and shape of the inlet port 112 may all be adaptively modified as required by the shape and size of the particular tubing assembly that the universal connector 100 is to secure. Some exemplar embodiments are illustrated and described below with regard to FIGS. 6 through 9.

For example, FIG. 6 presents a perspective view of an exemplar embodiment of an universal connector 100 consistent with the present invention that reveals a housing 120 with a consistent taper from the restrictor 110 to the outlet port 130. In the embodiment illustrated in FIG. 6, the inlet port 112 formed by the restrictor 110 is configured to contact the outer diameter of the first section of tubing 12 (FIG. 1) along a limited portion of the circumference of the tube. Moreover, the slot 122 formed by the housing 120 is slightly askew from the longitudinal axis of the universal connector 100. Such an arrangement of the slot 122 may serve to prevent inadvertent disengagement of the universal connector 100 from the underlying tubing assembly 10 (FIG. 1) that the universal connector 100 is designated to secure.

By way of further example, the schematic diagram of FIG. 7 presents the universal connector 100 of FIG. 4 in operative arrangement with the exemplar-tubing junction of FIG. 1. As illustrated in FIG. 7, the tapered male coupler 20 covered by a stretched portion of a tubing (i.e., the second tubing section 30 of the tubing assembly of FIG. 1) may be in close contact with the restrictor 110 of the universal connector to prevent forces applied along the first tubing section 12 from disengaging the partially enveloped tubing assembly. In addition, the tapered male coupler 20 covered by a stretched portion of a tubing may be in close contact with the universal connector 100 along the internal surface of the housing 120 such that forces applied along the second tubing section 30 (FIG. 1) do not result in disengagement of the protected tubing assembly 10 (FIG. 1)

The operative arrangement illustrated in the schematic of FIG. 7 reveals at least two structures of the universal connector 100 that may be configured such that they must be biased (i.e., forcibly deflected) in order to receive and/or release an appropriately sized tubing assembly designated for protection. A first example of a structure that may be manipulated to receive and/or release an underlying tubing assembly to be protected is the restrictor 110. As illustrated in FIG. 7, the restrictor plate 110 may be configured to receive a slightly oversized surface of a nipple associated with the tapered male coupler 20. An important relationship to realize this particular engagement is the relationship between the length of the gap between the two tab like protrusions of the restrictor 110 and the outer diameter of the nipple.

A second example of a structure or structures that must be manipulated to receive and/or release an underlying tubing assembly to be protected is the housing 120. As illustrated in FIG. 7, and as previously described the housing 120 may be configured to form a slot 122 for receiving and/or releasing a substantial portion of the tubing assembly 10. As illustrated in FIG. 7, the width of the slot 122 may be narrower than the diameter of tapered male coupler 20 with the over stretched second tubing section. This relationship necessitates the physical manipulation of one or both tapered portions of the housing 120 that surround the tubing assembly in order for the universal connector 100 to receive and/or release the tubing assembly.

While the present illustration and associated description reveals two locations where a universal connector 100 consistent with the present invention may be biased to receive and/or release a tubing assembly to be protected, a universal connector 100 is not limited to this particular arrangement. For example, the restrictor 110 may be modified such that it closely engages the first section of tubing 12 as opposed to the exterior of the nipple of the tapered male coupler 20. By way of further example, the slot 122 may be configured such that it is not necessary to bias both opposing surfaces of the housing 120 in order to engage and/or release the tubing assembly to be protected. In yet other embodiments, the restrictor 110 may be configured such that the inlet port 112 does not contact the nipple or the first section of tubing 12.

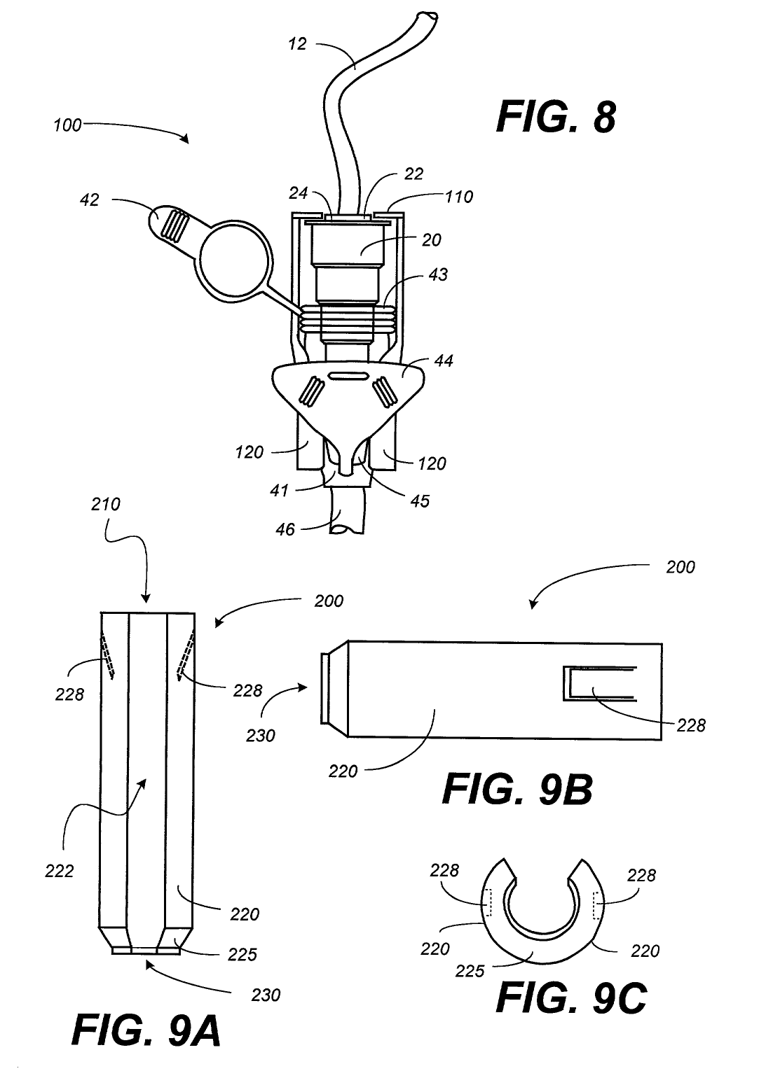

FIG. 8 is a schematic diagram illustrating another variation of the universal connector 100 of the present invention in operative arrangement with the exemplar-tubing junction of FIG. 3. In this regard, the universal connector 100 is configured with a housing 120 having a relatively short tapered portion shown herein just above the plug 44 of the protected tubing assembly. A universal connector 100 as shown in FIG. 8 may engage the Y-shaped fitting 41 along the tapered portion of the housing 120 such that forces applied to the second tubing section 46 do not result in disengagement of the junction formed by the Y-shaped fitting 41 and the tubing assembly 40 of FIG. 3. Note that in an alternative embodiment (not shown), the universal connector 100 may be configured with a second tapered portion substantially coinciding with the external surfaces of the Y-shaped fitting 41. For example, the housing 120 may be configured with a second tapered portion having a suitably configured diameter to closely receive and contact the base of the secondary input port 45 of the Y-shaped fitting 41. Alternatively, the housing 120 may be configured with a second tapered portion having a suitably configured diameter to closely receive and contact the base of the Y-shaped fitting 41. Still other embodiments may include a housing 120 having three or more tapered portions configured with diameters suited to receive and/or engage various structures of a tubing assembly to be protected.

FIGS. 9A-9C are a series of schematic diagrams illustrating another variation of the universal connector of the present invention in top, side, and front views, respectively. In this regard, FIG. 9A illustrates a top view of a universal connector 200 having a housing 220 having a tapered portion 225. As illustrated in FIG. 9A, a first non-tapered portion of the housing 220 may be configured to form an inlet port 210. Similarly, a second non-tapered portion of the housing 200 may be configured to form an outlet port 230. As further illustrated in FIG. 9A, the housing 220 may be configured with a slot 222 and with one or more restrictor tabs 228 (two shown). The inlet port 210, slot 222, tapered portion 225, and outlet port 230 generally function as in the previous illustrated and described embodiments of the universal connector.

The restrictor tabs 228 as illustrated in the various views presented in FIGS. 9A-9C may be adjusted to permit the insertion and/or release of a tubing assembly much like those previously illustrated and described in association with FIGS. 1 and 3. When in an operative or engaged configuration with a tubing assembly, the one or more restrictor tabs 228 may be biased inward to contact the leading edge of the nipple 22 and/or plate 24 of the tapered male coupler 24 common in the tubing assemblies 10, 40 illustrated in FIGS. 1 and 3, respectively. As with the previously illustrated and described embodiments of the universal connector 100 the tapered portion 225 of the housing 220 may be configured to engage the various structures of a tubing assembly such that forces applied along a second tubing portion (not shown) will not result in disengagement of the tubing assembly. In contrast with the previous embodiments of the universal connector 100, the universal connector 200 engages one or more surfaces of the tubing assembly to be protected via the one or more restrictor tabs 228 disposed along the interior of the universal connector 200. In this way, the universal connector 200 illustrated in FIGS. 9A through 9C may be configured to protect a suitably sized tubing assembly from disengaging due to forces applied along the first tubing section 12.

It should be appreciated that whereas the particular universal connector 200 illustrated in FIGS. 9A through 9C is depicted with two restrictor tabs 228 of substantially similar length, an universal connector in accordance with the teachings of the present invention is not limited to only this embodiment. For example, the interior surface of the housing 200 may be configured with three or more restrictor tabs 228 having one or more lengths as required to closely engage a tubing assembly to be protected. In addition, the multiple restrictor tabs 228 may be staggered across the inner surface of the housing 220 as required to engage any number of surfaces belonging to variously arranged tubing assemblies.

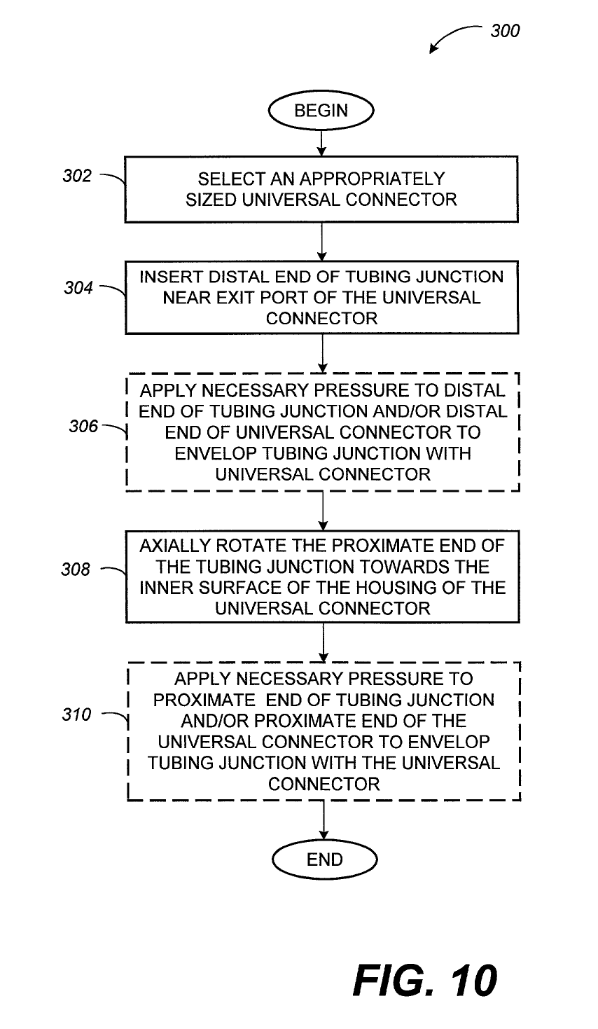

Reference is now directed to the flowchart of FIG. 10. In this regard, the flowchart presented in FIG. 10 illustrates a method 300 for securely coupling flexible tubing sections in a tubing assembly (e.g., tubing assemblies 10, 40 of FIGS. 1 and 3, respectively). As illustrated in FIG. 10, a medical technician, nurse, parent, patient, or other individual may select an appropriately sized and/or configured universal connector 100 as indicated in step 302. The technician or other individual practicing the method 300 may insert the distal end of a tubing assembly junction to be secured near an exit port of the universal connector as indicated in step 304. It will be appreciated that various configurations of the housing 120 (see FIG. 7) of the universal connector 100 may necessitate the manipulation or biasing of one or both portions of the housing 120 that form the slot 122.

Next, as illustrated in step 306, the technician practicing the method 300 may axially rotate the proximate end of the tubing assembly junction toward the inner surface of the housing adjacent an inlet port of the universal connector. As described above with regard to step 304, various configurations of the restrictor 110 (see FIG. 7) of the universal connector 100 may necessitate the manipulation or biasing of one or both tab like portions of the restrictor 110 that form the inlet port 112 in order to place the tubing assembly junction within the aperture 124 formed by the housing 120 (see FIG. 7). As described above, the universal connector 100 may be configured such that the technician biases the restrictor 110 to closely engage a portion of the first tubing section 12 (FIG. 7) and/or a portion of the nipple of the tapered male coupler 20 of the tubing junction.

Alternate implementations of the method 300 for securely coupling flexible tubing sections are included within the scope of the preferred method of the present invention in which functions may be executed out of order from that shown or discussed, including substantially, concurrently, or in reverse order, depending on the functionality involved, as would be understood by those reasonably skilled in the art of the present invention.

For example, those skilled in the art will recognize that a technician or other person practicing the method 300 for securely coupling flexible tubing sections in a tubing assembly may perform steps 304 and 306 in reverse order by inserting the inlet port of a tubing assembly near the proximal end of the universal connector and axially rotating the distal end of the tubing assembly junction to closely contact the inner surface of the housing of the universal connector near the outlet port of the connector. It will be further appreciated that the optional biasing steps described above may under some circumstances be performed in the reverse order or substantially simultaneously.

It should be emphasized that the above-described embodiments of the universal connector, particularly, any “preferred” embodiments, are merely possible examples of implementations, merely set forth for a clear understanding of the principles of the universal connector. Many variations and modifications may be made to the above-described embodiment(s) of the universal connector without departing substantially from the spirit and principles of the invention. All such modifications and variations are intended to be included herein within the scope of the universal connector as protected by the following claims.

Therefore, having thus described the invention, at least the following is claimed:

1. A connector, comprising:
means for receiving a first end of a tubing assembly junction;
means for engaging a substantial portion of the first end of the tubing assembly junction;
means for receiving a second end of a tubing assembly junction; and
means for securely coupling the tubing assembly junction wherein forces applied along the longitudinal axis of the tubing assembly do not result in disengagement of the tubing assembly at the junction.

2. The connector of claim 1, wherein the means for engaging comprises a housing.

3. The connector of claim 1, wherein the means for receiving a first end comprises a slot.

4. The connector of claim 1, wherein the means for receiving a second end comprises an aperture.

5. The connector of claim 1, wherein the means for securely coupling comprises a tapered inner surface of the housing.

6. The connector of claim 1, wherein the means for securely coupling comprises a restrictor.

7. The connector of claim 2, wherein the housing forms an aperture.

8. The connector of claim 3, wherein the slot is substantially parallel with the longitudinal axis of the connector.

9. The connector of claim 6, wherein the restrictor comprises a plate.

10. The connector of claim 6, wherein the restrictor comprises a tab.

11. The connector of claim 8, wherein the housing forms a slot having a width that is smaller than the outer diameter of an exit tube of the tubing assembly.

12. The connector of claim 9, wherein the plate forms an inlet port having a width that is smaller than the outer diameter of an inlet tube of the tubing assembly.

13. The connector of claim 9, wherein the plate forms an inlet port having a width that is smaller than the outer diameter of a nipple of a coupler of the tubing assembly.

14. The connector of claim 10, wherein the tab is biased into the aperture of the housing.

15. A method for securely coupling a tubing assembly, comprising:
selecting an appropriately configured universal connector;
inserting a first end of a tubing assembly junction within a housing of the universal connector; and
axially rotating a second end of a tubing assembly junction until the tubing assembly junction is substantially aligned with the longitudinal axis of the connector.

16. The method of claim 15, wherein the step of inserting a first end of a tubing assembly junction comprises substantially enveloping the outer circumference of a first tube of the tubing assembly.

17. The method of claim 15, wherein the step of inserting a first end of a tubing assembly junction comprises engaging an exterior surface of a structure of the tubing assembly junction with an inner surface of the connector.

18. The method of claim 15, further comprising:
biasing a structure of the housing of the connector to engage an outer surface of the tubing assembly junction.

19. The method of claim 18, wherein the structure comprises a restrictor.

20. A connector, comprising:
a housing having an inlet port, an outlet port, and a tapered inner surface, wherein the housing is configured to closely surround and contact a first end of a tubing junction; and
a restrictor fixedly attached to the housing, the restrictor configured to engage a second end of the tubing junction.
